(19) United States
(12) Patent Application Publication
(10) Pub. No.: US 2010/0034887 A1
(43) Pub. Date: Feb. 11, 2010

(54) Bursting Pellets

(75) Inventors: Polonca Kuhar, Ljubljana (SI); Peter Svete, Borovnica (SI); Judita Sirca, Ljubljana (SI)

Correspondence Address: ARENT FOX LLP, 1050 CONNECTICUT AVENUE, N.W., SUITE 400, WASHINGTON, DC, 20036, US

(73) Assignee: LEK Pharmaceuticals D.D., Ljubljana (SI)

(21) Appl. No.: 12/086,657

(22) PCT Filed: Dec. 21, 2006
(86) PCT No.: PCT/EP2006/012392

§ 371 Date: Oct. 7, 2009

(30) Foreign Application Priority Data
Dec. 23, 2005 (SI) P2005000354

(51) Int. Cl. A61K 9/14; A61K 31/415; A61P 19/02

(52) U.S. Cl. 424/489; 514/406

(57) ABSTRACT

In the present invention, a new pharmaceutical formulation of bursting pellets comprising in the core a high dose of a low potency active substance which is poorly soluble in water is described. Release of the active substance from the core takes place within minutes.

### FIELD OF THE INVENTION

The present invention belongs to the field of pharmaceutical technology and relates to a pharmaceutical formulation with a very fast disintegration.

More particularly, the invention relates to a pharmaceutical formulation of bursting pellets comprising in the core a high dose of an active substance which is poorly soluble in water. Release of the active substance from the core takes place within minutes.

### BACKGROUND OF THE INVENTION

There is a constant need for safe and conveniently administrable pharmaceutical formulations with immediate release of the active ingredient and rapid onset of therapeutic action.

Conventional formulations are usually cost-effective, relatively easy to produce and well-suited particularly to drugs for which an efficacy dose can be readily solubilized in the gastrointestinal tract (=GIT). Compounds amenable to this formulation design approach include either highly soluble drugs or highly potent and poorly soluble drugs for which the dose is sufficiently small to permit rapid and complete solubilization in the GIT.

Problems occur in the case of compounds with a poor aqueous solubility that require a high level of systemic exposure to achieve clinical efficacy.

Disintegrating pellets from a water-insoluble pectin derivative produced by extrusion/spheronisation have been shown superior, particularly regarding a faster release, over microcrystalline cellulose (=MCC) pellets for higher doses of low water soluble drugs, such as riboflavin (Tho I. et al., Eur. J. Pharm. Biopharm., 56 (2003) 371-80). Therein an insoluble polymer swells and consecutively the pellet disintegrates.

No reference could be found in patent and scientific literature from this field to solve the problem of providing a pharmaceutical formulation (particularly in pellet form) that would allow rapid release of high dosed and at the same time poorly water soluble active substances.

Thus the present invention is aimed at preparing an effective formulation for release within minutes of a high dosed and poorly water soluble active substance.

### SUMMARY OF THE INVENTION

In the first aspect, the invention concerns a pharmaceutical formulation comprising a pellet core from which a high dose of an active substance which is poorly soluble in water can be rapidly released.

In another aspect, the invention concerns a pharmaceutical formulation comprising a plurality of bursting pellets comprising at least one active pharmaceutical ingredient with a poor aqueous solubility and low potency and other pharmaceutical excipients that immediately disintegrate after the exposure to a dissolution medium

In another aspect, the invention concerns a pharmaceutical formulation which disintegrates to form a suspension within 5 minutes, preferably within 2 minutes and more preferably within 1 minute after exposure to the physiological fluid, water or other dissolution medium.

In another aspect, the invention concerns a pharmaceutical formulation having a specific surface area of the pellets larger than 0.5 m²/g, preferably larger than 1.0 m²/g, more preferably larger than 15 m²/g.

In another aspect, the invention concerns a pharmaceutical formulation comprising a pellet core comprising at least one excipient from each of the following groups: a gelling polymer, a filler and a surfactant.

In another aspect, the invention concerns a process for the preparation of such pharmaceutical formulations comprising preparation of the blend of the ingredients for the pellet core, granulation with demineralised water, extrusion and spheronization, drying, and filling into capsules or sachets.

In another aspect, the invention concerns a use of such pharmaceutical formulations with COX-2 inhibitors, particularly celecoxib, or pharmaceutically acceptable salts thereof for the treatment of osteoarthritis, rheumatoid arthritis or other diseases or disorders treatable with COX-2 inhibitors, either alone or in combination with other active principles.

### DETAILED DESCRIPTION OF THE INVENTION

Poorly soluble drugs with a low potency, which thus have to be administered in a high dose, are a big challenge for designing an immediate release formulation, since a rapid and complete solubilization of such drugs in GIT is aggravated. Poor aqueous solubility and slow dissolution rate frequently result in a low and erratic absorption, which especially for compounds with a narrow therapeutic index results in an unexpected toxicity or treatment failure.

In accordance to Biopharmaceutics Classification System (BCS), drugs are considered highly soluble when the highest strength (dose) is soluble in 250 mL or less of an aqueous medium over the pH range of 1.0-7.5. Otherwise, the drug substance is considered poorly soluble.

An immediate release drug product is characterized as a rapid dissolution product when not less than 85% of the labelled amount of the drug substance dissolves within 30 minutes using USP Apparatus I (Basket Apparatus) at 100 rpm or USP Apparatus II (Paddle Apparatus) at 50 rpm in a volume of 900 mL or Gastric Fluid (SGF) USP without enzymes; (ii) a pH 4.5 buffer; and (iii) pH 6.8 buffer or Simulated Intestinal Fluid (SIF) USP without enzymes. Otherwise, the drug product is considered to be a slow dissolution product.

In case, the active drug substance contributes a high percentage to the final dosage form composition, implementation of traditional solubility enhancing approaches is also limited.

The most appropriate among these techniques is known to be particle-size reduction, which effectively increases the surface area to volume ratio, thereby increasing the dissolution rate in the GIT and promoting absorption. Various technologies used for particle size reduction include micronization, nanonization and super critical fluid technology. Micronization, as a most commonly used method, may have two major disadvantages: aggregation, which often negates any improvement in dissolution, followed by particle size reduction and poor flow of milled particles that intensify difficulties during direct tabletting or capsule filling. Therefore, the formulation consisting of a micronized active substance, which maintains the positive effect of milling, such as a large specific surface area, and at the same time overcomes potential difficulties resulting from micronization, would be the ideal solution of the above mentioned problems.

We surprisingly found out that pellets, if composed by using a suitable excipient mixture, can be the perfect dosage form for immediate release of drugs with poor aqueous solubility and low potency. Due to their extremely short disintegration times such pellets were termed bursting pellets.

Bursting pellets have a specific composition which enables them to disintegrate to form a suspension within 5 minutes, preferably within 2 minutes and more preferably within 1 minute after exposure to the physiological fluid, water or other dissolution medium.

Due to an almost immediate increase of the contact surface, a fast dissolution of the active ingredient and therefore a rapid onset of therapeutic effect are enabled. Since the pellets are substantially round-shaped and of a relatively big diameter, such as 0.5 to 1 mm, they have an excellent flow and can be easily filled into capsules, such as hard gelatine capsules.

Such formulation (pellets) is particularly beneficial in case of particles that exhibit a poor flow either due to their shape (e.g. needle-shaped crystals) or physical properties of the particle surface (morphology, cohesiveness, electrostatics, . . . ).

Another advantage of bursting pellets over other currently available formulations is their porous surface, which enables a fast penetration of the dissolution medium into the pellet and consecutively an extremely short disintegration time of the pellets.

Furthermore, we have surprisingly found that bursting pellets have a large specific surface area which is comparable to the one of the micronized active ingredient contained therein. Specific surface area of the pellets means the sum of specific surface area of both the surface and pores inside the pellets. Large specific surface area of the pellets means that it is larger than 0.5 m²/g, preferably larger than 1.0 m²/g, more preferably larger than 1.5 m2/g.

Because of the large specific surface area of the pellets, the dissolution medium which penetrates into the pellet has a large contact surface area with the available active ingredient and immediately starts to dissolve it. Therefore the dissolution of the active ingredient in the pellet starts even before the pellet is disintegrated. Due to that fact, the dissolution of the active ingredient is even faster as it would be when using pellets with a smaller specific area and therefore a rapid onset of the therapeutic effect is accelerated.

Instead of an insoluble component, as reported in the prior art, a soluble excipient, i.e. a gelling polymer, is used. By this difference, the following effect is achieved: After a contact thereof with an aqueous medium, the polymer dissolution is initiated, thereby the pellet structure is destroyed and additional acceleration of the pellet disintegration is achieved.

Examples of drugs with poor solubility that could be used for the present invention include but are not limited to ziprasidone, raloxifene, phenobarbital, danazole, ketoconazole, sertraline, clarithromycin, and the like. The above described formulation is also suitable for COX-2 inhibitors, such as nimesulide, etoricoxib and especially suitable for celecoxib.

The amount of the drug in the pharmaceutical composition can be 50-90%, preferably 60-85%, more preferably 70-80%.

Thus the dose of the active pharmaceutical ingredient in such a pharmaceutical composition could be as much as 500 mg.

In addition to the active substance, the pellet core can comprise the following excipients: a filler, a gelling polymer (either of them can also have the function of an extrusion/spheronisation aid), a surfactant and optionally a binder.

Most commonly, microcrystalline cellulose can be used as an extrusion/spheronisation aid. It may be of any commercially marketed form, as well as silicified microcrystalline cellulose and the like. The amount of the extrusion/spheronisation aid can be at least 5%, preferably 6-10% and most preferably 8-15%.

The filler can be chosen among powdered cellulose, sorbitol, mannitol, various types of lactose, phosphates and the like. Preferably, lactose monohydrate can be used. The amount of the filler can be at least 2%, preferably 4-12% and more preferably 6-8%.

Surfactants may be non-ionic such as polyoxyethylene alkyl ethers, polyoxyethylene castor oil derivatives, polyoxyethylene sorbitan fatty acid esters (=polysorbate 80) and sorbitan monoesters, or ionic such as sodium docusate, docusate salts, sodium lauryl sulphate and the like. Preferably, sodium lauryl sulphate can be used. The amount of the surfactant can be 1-10%, preferably 3-7% and more preferably 4-6%.

Bursting effect of said formulation is due to gelling polymer. At least one of the following can be implemented: carboxymethyl cellulose sodium, carrageanan, hydroxypropyl cellulose, hydroxyethyl cellulose, hydroxypropylmethyl cellulose, alginic acid, sodium alginate, polyethylene oxide and the like. Preferably, carrageenan can be used. The total amount of the gelling polymer can be 1-20%, preferably 1-10% and more preferably 1-5%.

The binder can be selected among polyvinylpyrrolidone (=povidone), polyvinylpyrrolidone-vinyl acetate copolymer, alginic acid, guar gum, hydroxypropyl starch, pregelatinized starch, malto-dextrin, and the like. The portion of the binder can be less than 5%, preferably less than 3% and more preferably less than 1%.

Pellet cores can be prepared by processes conventional in pharmaceutical technology. For instance, the active pharmaceutical ingredient (e.g. celecoxib), microcrystalline cellulose, lactose, carrageenan and sodium lauryl sulphate can be combined and mixed. Sieved powder mixture can be granulated with demineralised water. From the homogeneous blend, pellet cores can be made using the method of extrusion and spheronization. Dried pellets can be filled into capsules of a suitable size, particularly hard gelatin capsules or HPMC capsules, or into sachets.

Optionally, the above described pellets can be additionally coated. Coating would be particularly beneficial in case of an acid-labile drug, since it would provide a complete protection of the pellet cores from the acidic gastric juice. After the transition of coated pellets into the small intestine, the coating would dissolve and bursting effect of pellets could take place. Coating could also be advantageous in case of drugs with a narrow absorption window where it would allow delivery to the absorption site followed by pH-triggered dissolution of coating and release of the active substance.

The pharmaceutical formulation according to the present invention comprising COX-2 inhibitors or pharmaceutically acceptable salts thereof can be used for the treatment of osteoarthritis, rheumatoid arthritis or other diseases or disorders treatable with COX-2 inhibitors, either alone or in combination with other active principles.

### EXAMPLES

The present invention is illustrated but in no way limited by the following examples:

### Example 1

| Ingredient | Amount [%] |
|---|---|
| Celecoxib | 74.0 |
| microcrystalline cellulose | 10.0 |
| lactose | 5.5 |
| carrageenan | 5.0 |
| sodium lauryl sulphate | 5.5 |

Celecoxib, microcrystalline cellulose, lactose, carrageenan and sodium lauryl sulphate are combined and mixed. Sieved powder mixture is granulated with demineralised water. From the homogeneous blend, pellet cores are made using the method of extrusion and spheronization. Dried pellets are filled into hard gelatin capsules, HPMC capsules or sachets.

### Example 2

| Ingredient | Amount [%] |
|---|---|
| Celecoxib | 74.0 |
| microcrystalline cellulose | 5.0 |
| lactose | 8.0 |
| carrageenan | 10.0 |
| sodium lauryl sulphate | 3.0 |

The method of preparation may be the same as with Example 1.

### Example 3

| Ingredient | Amount [%] |
|---|---|
| Celecoxib | 70.0 |
| microcrystalline cellulose | 10.0 |
| sorbitol | 10.0 |
| carboxymethyl cellulose sodium | 6.0 |
| polysorbate 80 | 4.0 |

The method of preparation may be the same as with Example 1.

### Example 4

| Ingredient | Amount [%] |
|---|---|
| Celecoxib | 80.0 |
| silicified microcrystalline cellulose | 8.0 |
| calcium phosphate | 5.0 |
| carboxymethyl cellulose sodium | 5.0 |
| sodium docusate | 2.0 |

The method of preparation may be analogous as with Example 1.

### Example 5

| Ingredient | Amount [%] |
|---|---|
| Celecoxib | 70.0 |
| microcrystalline cellulose | 10.0 |
| sorbitol | 8.0 |
| carboxymethyl cellulose sodium | 6.0 |
| polysorbate 80 | 4.0 |
| povidone | 2.0 |

The method of preparation may be analogous as with Example 1.

1. A pharmaceutical formulation characterised in that it comprises a plurality of bursting pellets that disintegrate after the exposure to a dissolution medium and comprise at least one active pharmaceutical ingredient with a poor aqueous solubility and a low potency, and other pharmaceutical excipients.

2. The formulation according to claim 1 which disintegrates to form a suspension within 5 minutes after exposure to the physiological fluid, water or other dissolution medium.

3. The formulation according to claim 2 which disintegrates to form a suspension within 2 minutes after exposure to the physiological fluid, water or other dissolution medium.

4. The formulation according to claim 3 which disintegrates to form a suspension within 1 minute after exposure to the physiological fluid, water or other dissolution medium.

5. The formulation according to claim 1 having a specific surface area of the pellets larger than 0.5 m²/g, preferably larger than 1.0 m²/g, more preferably larger than 1.5 m²/g.

6. The formulation according to claim 1 wherein said excipients comprise at least one compound from each of the following groups: a gelling polymer, a filler and a surfactant.

7. The formulation according to claim 1 wherein said active pharmaceutical ingredient is selected from the group of COX-2 inhibitors.

8. The formulation according to claim 7 wherein said active pharmaceutical ingredient from the group of COX-2 inhibitors is celecoxib.

9. A process for the preparation of pharmaceutical formulations according to claim 1 characterised in that it comprises the following steps: preparation of the blend of the ingredients for the pellet core, granulation with demineralised water, extrusion and spheronization, drying and filling into capsules or sachets.

10. Use of the pharmaceutical formulation according to claim 1 for the preparation of a medicament for the treatment of osteoarthritis, rheumatoid arthritis or other diseases or disorders treatable with COX-2 inhibitors.
